SEQUENCE LISTING (8 sequences)

SEQ ID NO: 1 - protein, 13 aa, Artificial
  biotin accepting peptide
LXXIXXXXKXXXX

SEQ ID NO: 2 - protein, 15 aa, Artificial
  biotin accepting peptide
GLNDIFEAQKIEWHE

SEQ ID NO: 3 - DNA, 366 bp, Artificial
  HBsAg-ScFv; antibody against HBsAg
atggccgaggtgcagctggtggagtctgggggaggcttggtcaagcctggagggtccctgagactctcctgtgcagactctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaagacacggccgtgtattactgtgcaagaaagctgaggaatgggaggtggcctctggtttattggggccaaggtaccctggtcaccgtgtcgaga

SEQ ID NO: 4 - DNA, 366 bp, Artificial; identical to SEQ ID NO: 3
  HBsAg-ScFv; antibody against HBsAg

SEQ ID NO: 5 - protein, 122 aa, Artificial
  HBsAg-ScFv; antibody against HBsAg
MAEVQLVESGGGLVKPGGSLRLSCADSGFTFSDYYMSWIRQAPGKGLEWVSYISSSGSTIYYADSVKGRFTISRDNAKNSLYLQMNSLRAEDTAVYYCARKLRNGRWPLVYWGQGTLVTVSR

SEQ ID NO: 6 - DNA, 378 bp, Artificial
  variable light chain domain
tctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggtgcggccgcagaacaaaaactcatctcagaagaggatctgaatggggccgcatag

SEQ ID NO: 7 - DNA, 378 bp, Artificial; identical to SEQ ID NO: 6
  variable light chain domain

SEQ ID NO: 8 - protein, 125 aa, Artificial
  variable light chain domain
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

(19) United States
(12) Patent Application Publication
(10) Pub. No.: US 2005/0048545 A1
(43) Pub. Date: Mar. 3, 2005

(54) Universal detection of binding

(76) Inventors: Millard Gambrell Cull, Brighton, CO (US); Miles Brennan, Denver, CO (US); Ronald Gill, Denver, CO (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/888,959

(22) Filed: Jul. 9, 2004

Related U.S. Application Data
(60) Provisional application No. 60/487,018, filed on Jul. 10, 2003.
(60) Provisional application No. 60/509,196, filed on Oct. 6, 2003.

(51) Int. Cl. C12Q 1/68; G01N 33/53

(52) U.S. Cl. 435/6; 435/7.1

(57) ABSTRACT

This invention relates to a universal detection system for ligand binding, and methods of use thereof. The universal detection system includes a Physically Alterable Binding Reagent and in some embodiments a Universal Detection Reagent.

### CROSS REFERENCE TO RELATED APPLICATIONS

This application claims the benefit of U.S. Provisional Patent application Ser. No. 60/487,018 filed Jul. 10, 2003, entitled “Universal Detection of Binding,” and also claims the benefit of U.S. Provisional Patent application Ser. No. 60/509,196, filed Oct. 6, 2003, entitled “Universal Detection of Binding.” Each of these applications is hereby incorporated by reference herein in its entirety.

### FIELD OF THE INVENTION

This invention relates to a universal detection system for ligand binding, and methods of use thereof. The universal detection system includes a Physically Alterable Binding Reagent and in some embodiments a Universal Detection Reagent. The Physically Alterable Binding Reagent includes a class of proteins having a ligand binding site, a domain that becomes physically altered upon binding of ligand to the ligand binding site, and, optionally, a sequence useful for coupling the binding reagent to a solid support. Binding affinity of the Universal Detection Reagent to the Physically Alterable Binding Reagent is altered upon ligand binding. Alternatively, the conformational change in the Physically Alterable Binding Reagent upon ligand binding can be detected by a physical method including but not limited to measurement of change in spectral quality, enthalpy, apparent molecular weight, surface area, and density that result from the conformational change. This invention is useful in any application where detection of ligand binding is desirable, such as diagnostics, research uses and industrial applications.

### BACKGROUND OF THE INVENTION

An antibody is frequently used to detect and quantitate levels of antigen. The binding properties of the antibody reagent in these assays typically impart a high degree of specificity and sensitivity. The challenge of these assays is to detect the binding event. There are several solutions in common practice. The most common method is the use of a “sandwich” assay. In this configuration, the capture antibody is immobilized on a surface and reacted with the unknown sample containing the antigen of interest. Following appropriate wash steps to remove unbound antigen, the bound component is detected using a second, antigen-specific antibody. Strategies to visualize the bound second antibody include using a second antibody that has been chemically coupled to a detection reagent, or using a third detection antibody (also chemically coupled to a detection reagent) that specifically recognizes and binds to the second antibody of the “sandwich” (e.g., using a mouse capture antibody, a rabbit second antibody, and a goat anti-rabbit Ig alkaline phosphate conjugated detection antibody). In the various iterations of this basic assay, the detection reagents for visualization of the bound antibody include for example, enzymes that react with substrates to give distinctive (e.g., colored) products, fluorescent dyes, or gold particles. The detection may also be by surface plasmon resonance (SPR), in which the increased mass of the bound second antibody is directly visualized on a surface by a change in manner in which it reacts with incident reflected light.

For detection of any particular antigen, this basic “sandwich” assay requires 1) two antigen specific antibodies (or proteins showing high, specific binding to ligands) that bind to a single antigen molecule in a non-interfering manner, and 2) means of detecting the bound second antibody/binding protein. The requirement for two antibodies that will simultaneously bind a single antigen molecule limits or complicates the use of this assay format in certain circumstances. It limits the use of this format for detection of antigens, including small monovalent haptens and small peptides (e.g., small peptide hormones). It also complicates the use of this general format for use in antibody-array proteomics chips in which the presence of large numbers of antigens are being simultaneously detected. In this case, the need for an antigen-specific second antibody doubles the number of antibody reagents that must be developed.

Presently, the identification of antigen:antibody complexes has taken a number of different forms. As non-limiting examples, 1) an antibody is immobilized and allowed to react with a sample. Bound antigen is then detected by binding of a second, labeled, molecule such as a ligand for the antigen or another antibody directed against another epitope of the antigen; 2) an antibody is immobilized and then allowed to react with a sample. The occupancy of the antigen binding sites by antigen from the sample is determined by a subsequent or concurrent reaction with labeled antigen; 3) an antibody is immobilized on a substrate such as a slide and then allowed to react with a sample. The antigen:antibody complex is detected by a method such as surface plasmon resonance; 4) an antibody in solution is reacted with a sample and with a labeled ligand. The amount of antigen displaces labeled antigen, and the amount of antigen in the sample is reflected in the decreased polarization; and 5) all components of a sample are chemically labeled (e.g., with a fluorescent dye such as Cy3 or Cy5), and then allowed to react with the immobilized antibody. Antigen binding to specific antibody spots is assessed by fluorescence. These techniques all exploit either an antigen specific reagent and/or the molecular weight of the antigen:antibody complex relative to antigen or antibody alone.

For these applications in particular, and for general use with solid phase immunoassays, it would be a significant benefit to utilize a single “universal” reagent or physical method that detects antigen-antibody binding and that does so in a manner that is not specific for the particular antigen involved.

Most vertebrates produce several isotypes of immunoglobulin (e.g., IgM, IgG, IgA, IgD, IgE) that differ by their heavy chain constant region and have specialized biological properties. The basic immunoglobulin structural unit is composed of four peptide chains, two identical heavy chains and two identical light chains, forming a Y-shaped molecule. Each unit contains two antigen combining sites (one at each tip of the “Y”). Additional domains on the stem of the “Y” mediate various effector functions such as Fc-receptor binding and complement component Clq binding that leads to complement activation via the classical pathway.

IgM is found typically as a pentameric molecule composed of five IgG-like subunits described above, plus an additional single peptide, the J-chain. The five subunits and J-chain are held together in the pentamer by inter-chain disulfide bonds. Monomeric and higher multimeric forms, including hexamers, have been observed. Multimeric forms with and without the J chain have been observed. The pentamer forms a flat planar molecule in solution in the absence of bound antigen. Upon binding specific antigen, there is a well-documented dramatic conformational change in the molecule that now assumes a “staple” configuration, with nearly a 90 degree angle formed between the Fab and Fc portions of the monomer subunit. This conformational change has important biological consequences that are exploited in this invention. In particular, the affinity of Clq binding to IgM changes in response to antigen binding. The conformational change alters the accessibility of the J-chain of IgM for interaction with anti-J chain specific antibody. This invention demonstrates the utility of using this IgM conformational change, as revealed by a conformation-dependent, but specific antigen-independent detection reagent (e.g. a Clq- or Clq binding site- or anti-J chain-specific antibody) to detect IgM bound to its specific antigen. Further, the dramatic conformational change in IgM induced by antigen binding can also be detected by physical methods. These can be detected by a physical method including but not limited to measurement of change in spectral quality, enthalpy, apparent molecular weight, surface area, and density that result from the conformational change.

Much of the literature describing the interaction of IgM with its specific antigen and the resulting conformational changes have used multivalent antigens (one molecule or particle containing multiple antigenic sites that react with the specific IgM under investigation). There is limited data available for interaction with monovalent antigen or haptens. Nevertheless, there are data that support a compelling argument that 1) interaction of multimeric IgM with hapten or monovalent antigen (the relevant configuration for most proteomics applications, for example) is sufficient to induce an IgM conformational change, as indicated by, e.g., Clq binding or reaction with anti-J chain specific antibody, 2) “monomeric” IgM (IgMs constituting a single IgG-like subunit described above, composed of the two identical Ig mu heavy chains and two identical Ig light chains, but lacking the J-chain and not assembled into a multimer) also binds Clq in an antigen-dependent fashion and so presumably also undergoes a conformational change analogous to that of the multimer, 3) little or no Clq binding or reaction with anti-J chain specific antibody is detected with IgM or IgMs in the absence of antigen (in contrast to unaggregated IgG).

### SUMMARY OF THE INVENTION

The present invention relates to a general, universal binding detection system, and methods of use thereof. One embodiment of the Invention requires at least two reagents: 1) a Physically Alterable Binding Reagent, and 2) a Universal Detection Reagent. The Physically Alterable Binding Reagent includes a class of proteins having a ligand binding site, a domain that becomes physically altered upon binding of ligand to the ligand binding site, and, optionally, a sequence useful for coupling the binding reagent to a solid support. Binding affinity of the Universal Detection Reagent to the Physically Alterable Binding Reagent is altered upon Physically Alterable Binding Reagent- ligand binding. The Universal Detection Reagent binds to the Physically Alterable Binding Reagent. The Universal Detection Reagent exhibits altered affinity for the Physically Alterable Binding Reagent after ligand binding by the Physically Alterable Binding Reagent. Optionally, the Universal Detection Reagent has a reporter moiety. Further, the system is generally applicable to binding of ligand to any binding protein for which there is a conformational change upon binding, and a reagent specific for that change.

In a second embodiment, the Physically Alterable Binding Reagent undergoes a conformational change that can be directly detected by physical means without the use of a second detection reagent. This embodiment is useful when the ligand is small and not amenable to a sandwich type assay.

In a one embodiment the Physically Alterable Binding Reagent is an antibody fusion and the ligand is its cognate antigen, in which case the invention is a general system for detecting antigen-antibody binding events. The system is based on detection of conformational changes that normally occur when certain antibody molecules bind to their cognate specific antigen. This response is particularly dramatic for IgM, but may also occur to a significant extent with other isotypes. Changes in antibody conformation upon antigen binding is detected by various physical means or by the altered affinity of complement component Clq to the complex or by other proteins for which the interaction with antibody is conformation-dependent e.g., conformation-specific antibody reagents and (naturally occurring or engineered) antibody binding proteins such as Fc-receptors and related molecules.

This invention is useful in any application where detection of ligand binding is desirable, such as diagnostics, research uses and industrial applications. This method is particularly well suited to detecting antigen-antibody complexes on either protein- or antibody microarrays, although fluid phase applications using soluble components are also envisioned.

A novel aspect of this invention is that the conformational change can be measured directly by physical means or a single reagent can be developed that will detect antigen-antibody binding for multiple (if not all) antibody species (at least for antibodies of the IgM isotype) irrespective of their individual antigen specificity.

The present invention also encompasses methods of use of the above-described system.

### BRIEF DESCRIPTION OF THE DRAWINGS

FIG. 1 shows the AnitHBsAg pcDNA5 vector. Anti-HBsAg IgM heavy chain: base pairs 898-2724. Anti-HBsAg IgK light chain: base pairs 3411-4163.

FIG. 2 shows a schematic presentation of the Universal Detection System using Viral Chips. The AviTagged IgM against HIV (column A), HBV (column B), HCV (column C) and SARS (column C) will arrayed in duplicate (rows 1 and 2) or total IgM isolated from normal human blood (row 3) which will serve as a control spot for each column. Each group of arrays on the slide (rows 1-3) will be mounted with a leak-proof plastic divider which will create wells similar to ELISA plates. The test and normal sera will be loaded and the virus will be captured by specific antibodies. The conformational change in IgM occurs due to binding of the viral particles This change will be detected by the universal detection reagent.

### DETAILED DESCRIPTION OF THE INVENTION

The invention provides a general method for detecting ligand binding, particularly antigen:antibody complexes. The system of the invention involves direct measurement of the conformational change in the Physically Alterable Binding Reagent by physical means or by use of a Universal Detection Reagent. In an embodiment of the invention, the system comprises two reagents: 1) a Physically Alterable Binding Reagent, and 2) a Universal Detection Reagent.

The following terms are intended to have the following general meanings as they are used herein.

### A. Definitions

“Physically Alterable Binding Reagent” or “Binding Reagent” includes a class of proteins having a ligand binding site, a portion that becomes physically altered upon binding of ligand to the ligand binding site, and, optionally, a sequence useful for coupling the binding reagent to a solid support, also known as a tag. The Physically Alterable Binding Reagent can be any molecule that binds a ligand specifically and becomes physically altered as a result of such binding, including, but not limited to, antibodies or receptor-binding domains. The Physically Alterable Binding Reagent is typically a modular molecule, including but not limited to fusion (“chimeric”) molecules, having a ligand binding site and a portion that becomes physically altered upon binding of ligand to the ligand binding site. In general, this molecule may be naturally occurring or may be made through genetic engineering, but other methods that accomplish the same goals are contemplated. The ligand binding site includes the range of specificities of immunoglobulin molecules. An embodiment of a ligand binding site is an antigen combining site contained with the variable region of an immunoglobulin molecule or Fab sequences of immunoglobulin molecules. The portion that undergoes physical alteration upon ligand binding can include a wide variety of those currently known in the art, including but not limited to, portions of a transmembrane ligand specific receptors and Immunoglobulin M (IgM). One embodiment of a portion that undergoes physical alteration upon ligand binding is the constant region of the IgM molecule. Upon ligand binding, the IgM molecule undergoes a physical change that exposes a binding site for the complement factor Clq. For the purposes of this invention, IgM can be monomeric or multimeric, including pentamers and hexamers with or without associated J chain. Further, the IgM may be natural, modified, or engineered.

“Universal Detection Reagent” or “Detection Reagent” binds the Physically Alterable Binding Reagent after ligand binding and optionally, has a reporter moiety. As a non-limiting example, the binding of ligand to IgM alters Clq binding affinity and in this case, Clq would be a substantial portion of the Universal Binding Reagent. The Clq can be coupled to any number of reporter moieties, including but not limited to, fluorescent reporter molecules, enzymes, and nanobeads. Other non-limiting examples of Universal Detection Reagents include those where the binding of ligand to various receptors results in the activation or inactivation of physically distinct sites for binding or for enzymatic reaction, such as a kinase activity: the Universal Detection Reagent is a substrate or ligand for the Physically Alterable Binding Reagent at this physically distinct site. Alternatively, the Universal Detection Reagent can be an antibody specific for a conformation dependent epitope of the Physically Alterable Binding Reagent.

“Measurement by physical means or methods” is any physical measurement that can detect the change in the Physically Alterable Binding Reagent upon binding of its ligand. The physical means include, but are not limited to, a shift in the absorbance or emission spectra or light scattering and transmission behavior or other methods where the conformational change between the Reagent with and without bound ligand can be detected by means of an altered interaction with electromagnetic radiation. Such physical methods that are currently in use that can detect conformational changes in the Physically Alterable Binding Reagent include, but are not limited to, fluorescence emission spectra including second derivative measurements and Wood's anomaly, Raman shift spectroscopy, Fluorescence Resonance Energy Transfer (FRET), fluorescence quenching, and Surface Plasmon Resonance. Other methods that measure changes in density, apparent molecular weight, or surface area including but not limited to Atomic Force Microscopy could be used to detect the conformational change in the Physically Alterable Binding Reagent.

“Ligand” is any molecule that is capable of being captured in a binding site. An antigen is the ligand for an antibody. Ligands are typically molecules that it is desirable to measure in various applications and can include proteins, peptides, small molecules, carbohydrates, drugs and the like.

“Antibody” or “Ab” or “Immunoglobulin” is a protein that binds specifically to a particular antigen and is capable of selectively binding to at least one of the epitopes of the protein or other antigenic substance used to obtain the antibodies and is derived wholly or in part from the immunoglobulin coding regions of the respective animal. Antibody molecules differ in their specificity by virtue of variability in the amino acid sequence of their “variable region domains”. Antibodies useful in the present invention can be either polyclonal or monoclonal antibodies. Antibodies of the present invention include functional equivalents such as antibody fragments and genetically-engineered antibodies, including single chain antibodies. Antibodies of the present invention also include chimeric antibodies that can bind to more than one epitope.

“Tag” means any domain on one molecule that facilitates its association with another molecule. In one embodiment, the tag is a peptide sequence that can be expressed as part of a Physically Alterable Binding Reagent, including an antibody, that can serve to immobilize the Physically Alterable Binding Reagent to a solid support. Additionally, the tag can be a chemical group that can be used for chemical immobilization in one embodiment, the peptide sequence can encode a peptide tag for the recognition sequence for enzymes for associating non-proteinaceous molecules such as biotin or carbohydrates or any other post-translational modification of the protein. The association can be either covalent or non-covalent. The tag can be any peptide sequence that has these properties. A number of peptide sequences that have the properties of a Tag are known and can be used in the present invention. As a non-limiting example, the following peptide sequences can be tags of the present invention: a biotin accepting peptide sequence, hexa-His peptide, Strep-Tag, Strep-TagII, FLAG, c-myc, maltose binding protein (MBP), glutathione-S-transferase (GST), green fluorescent protein (GFP), red fluorescent protein (RFP), blue fluorescent protein (BFP), chitin binding protein, calmodulin binding protein (CBP), cellulose binding domain, S-tag, FIAsH, RsaA, and other similar types of peptide sequences having the ability to facilitate association with another molecule. In one embodiment of the invention, antibodies include as part of their coding sequences a biotin accepting peptide sequence that is a short amino acid sequence discovered by Schatz that allows the enzymatic attachment of biotin to a lysine residue within the tag. The Schatz biotin accepting peptide sequences are described in U.S. Pat. No. 5,723,584 issued on Mar. 3, 1998, U.S. Pat. No. 5,874,239 issued on Feb. 23, 1999, U.S. Pat. No. 5,932,433, issued on Aug. 3, 1999 and U.S. Pat. No. 6,265,552, issued Jul. 2001. In general, biotin accepting peptide sequences have the following sequence: LeuXaa₁Xaa₂IleXaa₃ Xaa₄Xaa₅Xaa₆LysXaa₇Xaa₈Xaa₉Xaa₁₀, (SEQ ID NO: 1) where Xaa₁ is any amino acid; Xaa₂ is any amino acid other than Leu, Val, Ile, Trp, Phe, or Tyr; Xaa₃ is Phe or Leu; Xaa₄ is Glu or Asp; Xaa₅ is Ala, Gly, Ser, or Thr; Xaa₆ is Gln or Met; Xaa₇ is Ile, Met, or Val; Xaa₈ is Glu, Leu, Val, Tyr, or Ile; Xaa₉ is Trp, Tyr, Val, Phe, Leu, or Ile; and Xaa₁₀ is any amino add other than Asp or Glu, wherein said biotinylation-peptide is capable of being biotinylated by a biotin ligase at the lysine residue adjacent to Xaa₆.

One embodiment of a biotin accepting peptide sequence is Gly Leu Asn Asp Ile Phe Glu Ala Gln Lys Ile Glu Trp His Glu (SEQ ID NO:2), and this sequence is referred to as the AviTag™. The peptide tag may, optionally, be appended to any part of the Physically Alterable Binding Reagent. In one embodiment, the peptide tag is incorporated into a constant region of an immunoglobulin locus that is part of the Physically Alterable Binding Reagent. Alternatively, the Physically Alterable Binding Reagent can be covalently attached to any solid support without a Tag.

“Solid support” includes any suitable support for a binding reaction and/or any surface to which molecules may be attached through either covalent or non-covalent bonds. This includes, but is not limited to, membranes, plastics, paramagnetic beads, charged paper, nylon, Langmuir-Blodgett films, functionalized glass, germanium, silicon, PTFE, polystyrene, gallium arsenide, gold and silver. Any other material known in the art that is capable of having functional groups such as amino, carboxyl, thiol or hydroxyl incorporated on its surface, is also contemplated. This includes surfaces with any topology, including, but not limited to, flat surfaces, spherical surfaces, grooved surfaces, and cylindrical surfaces e.g., columns. Multiple Physically Alterable Binding Reagents, each specific for a different ligand, may be attached to specific locations on the surface of a solid support in an addressable format to form a an array, also referred to as a “microarray” or as a “biochip.”

### B. The General Method

One embodiment of the invention is composed of two compound reagents: an IgM or IgM-like Physically Alterable Binding Reagent and a Universal Detection Reagent (such as Clq) that detects the conformational change of the Physically Alterable Binding Reagent. The Physically Alterable Binding Reagent is made as follows. Where the Physically Alterable Binding Reagent is an antibody, antibody fragment, or antibody-like molecule, that can be prepared by any method known in the art, such as the preparation of monoclonal antibodies via the hybridoma method, the use of antibody phage display libraries, and so on. When the antibody is an IgM expressed by a hybridoma or B-cell, the antibody can be purified from this source directly. When the antibody is a non-IgM expressed by a hybridoma or B-cell, then the segment of the cDNA encoding the antibody variable regions are cloned into an expression vector such that the variable regions are operably linked to the coding sequence for the portion of the Physically Alterable Binding Reagent that can undergo conformational alteration, such as the IgM constant region. The expression vector is introduced into a cell line and the Physically Alterable Binding Reagent, such as chimeric IgM is purified. The purified chimeric IgM is tested to confirm that that the recombinant IgM retains the expected antigen specificity and affinity. By “specificity,” it is meant that the IgM selectively binds the specified protein or other antigen. Binding can be measured using a variety of methods known to those skilled in the art including immunoblot assays, immunoprecipitation assays, radioimmunoassays, enzyme immunoassays (e.g., ELISA), immunofluorescent antibody assays and immunoelectron microscopy. Antibodies that exhibit a specific binding at a level suitable for detection can be used as the Physically Alterable Binding Reagent.

In this embodiment, the Physically Alterable Binding Reagent is immobilized to a solid support and the test fluid suspected of containing the antigen of interest is added. If the antigen of interest binds to the Physically Alterable Binding Reagent, the IgM portion of that molecule changes conformation and is detectable either by physical means, by its ability to bind Clq, or by its ability to bind another molecule that recognizes the conformational change. In this case, the Universal Detection Reagent is Clq with an attached reporter molecule. The Universal Detection Reagent is added and the amount of bound reporter molecule is detected.

The method presented here includes the measurement of a physical change in an receptor molecule induced by the binding of ligand. Such a change would be independent of the nature of the ligand, and thus suited for simultaneous measurements of different receptor:ligand (antigen:antibody) complexes. For example, when a number of different antibodies are immobilized on a slide and allowed to react with a sample, the antigens in this sample bind to their cognate receptors/antibodies immobilized on different regions of the slide. These antibodies with bound antigen can then be detected by allowing them to react with a second reagent that binds specifically to receptors/antibodies with bound ligand/antigen.

This embodiment of the invention provides a general method for detecting antigen-IgM antibody binding events. The method is independent of the specific antigen used, and relies instead on the general characteristic of IgM in which binding of specific antigen results in a conformational change in the IgM molecule, and that this change can be detected using reagents such as the complement component, Clq; anti-J chain specific antibody; or an antibody that recognizes the conformational change.

The basic form of this invention is detection of specific antigen binding to its cognate IgM antibody when the antibody is immobilized as derivatized areas on a surface such as a glass slide. IgM is immobilized on the slide using existing or adapted technologies. One embodiment of the method would use a genetically engineered attachment linker or tag that would allow the antibody to be tethered in a consistent orientation and allow sufficient flexibility for the antibody to undergo its conformational changes upon antigen binding or hinder the binding of the detection reagents. For example, an IgM genetically engineered to contain an AviTag and an extended flexible linker to the C-terminus of the Ig heavy chain will allow for robust, yet flexible tethering of the IgM to streptavidin coated surfaces.

The slide containing the immobilized IgM antibody is then reacted with the antigen-containing test material, followed by appropriate washing to remove unbound material. Molecules of immobilized antibody that have bound specific antigen will have undergone the characteristic conformational change associated with antigen binding.

In this method, the binding of antigen to the immobilized IgM is detected indirectly by virtue of the conformational change in the antibody structure. In this iteration of the method, the antibody conformation is detected by the differential binding of Clq (or Clq derived molecule) to the antigen-bound conformation of the IgM.

Immobilized antibody for which there is cognate antigen in the test material will bind the cognate antigen and the antibody will become conformationally altered. Antibody spots for which there is no cognate antigen in the test material fail to bind antigen and are not conformationally altered. Differential Clq binding can be used to distinguish those antibodies that have undergone conformational change from those that have not.

Detection of Clq bound to antibody antigen-antibody complexescan be accomplished by one of several possible methods For example, the Clq detection reagent may be a Clq fluorescent conjugate (e.g., fluorescein, Cy3, Cy5, etc.) which would generate a fluorescent signal, or the altered refractive index associated with the immobilized antibody can be detected directly by SPR. Alternatively, Clq bound to antigen-antibody complexes may be detected by FRET technology. Clq binding can be similarly visualized by addition of a polyclonal anti-Clq antibody (e.g., by fluorescence using a fluorescent conjugated anti-Clq antibody, or unconjugated anti-Clq antibody with SPR detection). The latter method generates an amplified signal since polyclonal anti-Clq antibody contains several antibody species that recognize and bind simultaneously to different epitopes on the Clq molecule.

### C. Making the Physically Alterable Binding Reagent

The method of the invention is based on a conformational change in a binding reagent upon binding to its cognate ligand, particularly the conformational change in an antibody upon binding to its cognate antigen. In one embodiment the Physically Alterable Binding Reagent is an IgM or IgM-like molecule. Suitable IgM can be obtained from any one of several sources. Polyclonal IgM from immunized animals can be obtained from serum or secretions, and purified by affinity purification on the basis of the mu chain and/or antigen specificity. These naturally occurring IgM molecules undergo a suitable conformational change that is readily detected by differential binding of Clq.

The predominant Ig types from hyperimmune serum or by monoclonal antibody-producing hybridomas are IgG. General methods for making suitable IgM Physically Alterable Binding Reagent reagents from these sources are described herein. IgM producing cell lines can be produced de novo, e.g., from immunized animals by standard cell fusion techniques or by immortalization of IgM producing B-cells with Epstein-Barr virus, and the resulting cell lines screened for those producing IgM with the desired antigen specificity. In one embodiment, existing hybridomas producing Ig with an antigenspecificity of interest, but of an isotype other than IgM, can be used to create cell lines producing chimeric IgM having the Ig heavy- and Ig light- chain variable regions (and therefore the antigen binding affinity and specificity) derived from the original Ig molecule and IgM-derived mu-heavy chain constant regions. For example, in one embodiment, MRNA from an IgG-producing hybridoma, is used in RT-PCR to amplify the heavy and light- chain variable regions of the expressed Ig light and heavy chains, and cloned into respective light chain and mu heavy chain expression vectors. The vectors contain an intact cDNA of the kappa (or lambda) light chain, or the mu heavy chain constant regions, such that the respective variable regions of each chain can be appended to other respective constant regions. The resulting constructs are co-expressed (with or without expression of the J-chain) in appropriate cell lines (e.g., non-Ig producing myeloma) for expression. Alternatively, the IgG (or other) producing hybridoma can be manipulated genetically to produce IgM with the same variable regions and having the same antigen specificity and affinity. This is accomplished by targeted gene replacement in which the mu heavy chain constant region is inserted in the chromosome juxtaposed to the respective rearranged and expressed heavy chain variable region gene. The resulting construct expresses a chimeric IgM having a heavy chain that is derived from (a) the original variable region and (b) the introduced mu constant regions. An Example of this process is described in Example 6. In this illustration, the Hepatitis B virus surface antigen (HBV-sAg) binding regions from a mouse IgG-producing hybridoma are used. This method can also utilize the variable regions or ligand-binding domains derived from any class of antibody (e.g., IgY, IgG, IgM, IgA, IgE,), or immunoglobulin-like cell surface receptor molecules (e.g., T-cell receptors and other cell surface receptor molecules), or other ligand-binding proteins, from a wide array of mammalian species (including but not limited to goats, rabbits, mice, rats, horses, llamas), or from avian species such as chicken, or from fish species (including but not limited to sharks and zebrafish).

Antibody-like molecules, such as single-chain variable region fragment (scFv) antibodies from phage display, may also be “converted” to authentic IgM suitable for use with this invention and having the antigen specificity of the original antibody-like molecule. For example, the respective variable regions of a single chain antibody gene of interest may be cloned and inserted into appropriate light chain and mu heavy chain expression vectors as described in Example 7.

While Example 6 describes the use of the Flp-In™ vector from Invitrogen, this method may use any technique known in the art to introduce DNA of the expression vector into cells (e.g., transfection or infection with viral vector). “Cells” include eukaryotic cells, prokaryotic cells, and archae. Alternatively, an expression vector capable of replication and stable maintenance in the cellular cytoplasm and not requiring chromosomal integration could have been used to express the IgM Universal Detection reagent. Examples of this type of vector include, but are not limited to, Bovine Papilloma Virus expression or a vector using a Hepatitis E virus replicon.

The parental vector would contain the IgM heavy constant regions to which various binding domains from immunoglobulin molecules from various species including, but not limited to sharks and other fish, mammals including horses, donkeys, llamas, goats, pigs, rodents and rabbits, avian species such as chickens, and for receptor molecules, especially immunoglobulin-like receptors such as the T-cell receptor.

IgM may be expressed in a variety of configurations (e.g., various oligomeric forms, including monomers, pentamers, hexamers, and forms with or without the associated J-chain, and forms with or without an associated “secretion component” derived from the polymeric immunoglobulin receptor, and membrane-bound surface form of IgM, sIgM, or the soluble form of IgM). In certain applications, one or another of these forms may be most applicable. While in most cases, the constant regions of antibody molecules do not appear to undergo physical change (allosteric or conformational) upon antigen binding, IgM is an exception: IgM is a pentamer (or in some cases a tetramer or hexamer) of dimers each with an antibody combining site of one heavy and one light chain, all joined by a “J-chain”. Thus, these molecules are decavalent for antigen combining sites. The best evidence indicates that occupancy of two or more antigen binding sites on this molecule induces a change such that binding sites for complement factor Cl (or sub-factor Clq) are exposed. As the affinity of IgM for antigen is generally low, occupancy of two or more antigen binding sites normally occurs only when the antigen is multivalent. However, it is possible to select for IgM of high affinity or to introduce antigen combining sites (variable regions) of high affinity from other classes of immunoglobulins into IgM by means of recombinant DNA techniques. The cell line, including a mutanagized hybridoma cell line, engineered to produce the IgM Physicially Alterable Binding Reagent can be made with or without the J chain. The J chain could be produced by co-transfection of the J-chain producing vector with the IgM expression vector, could be co-expressed a different promoter on the expression vector that produces the IgM, could be produced by a combination of chromosomally intergrated and cytoplasmic expression replicon, or by any method that is currently used to express multiple or multi-subunit proteins. Additionally,

### D. Direct Physical Means of Detecting the Conformational Change

Any physical parameter that varies with the conformational change of the Physically Alterable Binding Reagent upon binding of its ligand may be used as a measure of ligand. The physical parameters include, but are not limited to, a shift in the absorbance or emission spectra or light scattering and transmission behavior or other methods where the conformational change between the Reagent with and without bound ligand can be detected by means of an altered interaction with electromagnetic radiation. Physical methods that are currently in use that can detect conformational changes in the Physically Alterable Binding Reagent include, but are not limited to, fluorescence emission spectra including second derivative measurements and Wood's anomaly, Raman shift spectroscopy, Fluorescence Resonance Energy Transfer (FRET), fluorescence quenching, and Surface Plasmon Resonance. Other methods that measure changes in density, apparent molecular weight, or surface area including but not limited to Atomic Force Microscopy could be used to detect the conformational change in the Physically Alterable Binding Reagent.

### E. The Universal Detection Reagent

In this invention, ligand binding is detected indirectly by a conformational change in the structure of the Physically Alterable Binding Reagent. The conformational change in the Physically Alterable Binding Reagent may be detected by direct physical means (section D, above) or by use of the “Universal Detection Reagent”. In one embodiment, ligand binding is detected indirectly by a conformational change in the structure of an IgM molecule. As described above, the complement component Clq exhibits differential binding affinity for the antigen-bound and unbound forms of IgM. Therefore, Clq, the Ig binding domain of Clq, or any other molecule with similar binding properties and specificity are reagents that can be used as the Universal Detection Reagent in this method. Clq may be purified from the serum of vertebrate animals or expressed by recombinant genetic methods; the Clq Ig binding domain may be derived from the intact protein by proteolytic cleavage or by expressed recombinant genetic methods as an isolated domain or fused to a carrier protein. Other types of molecules that may be used as detection reagents include, for example, peptides, nucleic acids and antibodies (natural or recombinant antibody-like molecules including scFv derived from phage display methods) that exhibit differential binding affinity for the antigen-bound and unbound forms of IgM.

An alternative Universal Detection Reagent is anti-J chain antibody. Some forms of IgM contain an additional peptide, the J-chain. The J-chain exhibits differential accessibility to anti J-chain antibodies in the antigen-bound and unbound forms of IgM.

An alternative detection is by fluorescent resonance energy transfer. In this iteration of the method, the IgM is engineered to contain appropriate fluorophores on opposite sides of the bend that occurs in the IgM upon antigen binding. The choice of fluors (e.g., GFP, related fluorescent proteins and their spectral variants) is such that the first fluor has a unique excitation wavelength, and its emission spectra matches the excitation wavelength of the second fluor. This phenomenon occurs to a measurable extent only when the two fluors are closely juxtaposed to one another, i.e., in this case, when IgM has undergone its characteristic conformational change thereby bringing the two fluors into closer proximity. Readout is by detection of light at the emission wavelength characteristic of the second fluor.

Clq may be used as a Universal Detection Reagent for IgM-derived Physically Alterable Binding Reagent molecules. The IgM molecule may be engineered such that the amino acid sequence of the Clq binding site has been altered. In this IgM, the conformational change occasioned by antigen binding uncovers the altered site. This novel site may be engineered to be the binding site of another reporter molecule, distinct from Clq,but functionally analogous with respect to its use as a Universal Detection Reagent. Alternatively, the altered site may have catalytic activity or serve as an enzyme substrate, where these activities are dependent upon the conformational state (ie. ligand bound or un-bound state) of the Physically Alterable Binding Reagent.

### F. Detection

Any suitable detection system is envisioned for quantitation of Universal Detection Reagent binding in the present invention, including but not limited to fluorescence, enzyme coupling, radioactivity, surface plasmon resonance, chemiluminescence, and the like. Several means are envisioned which will allow the binding of the Clq detection reagent (or binding of any other conformation-dependent molecule) to the IgM-antigen complex to be detected. These include coupling the detection reagent (e.g. Clq) to an enzyme (e.g., alkaline phosphatase or peroxidase) or to a fluorophore. Visualization is by reacting with an appropriate colored or fluorescent substrate, or by direct visualization of fluorescence, respectively. The signal may be amplified by reacting with the detection reagent (e.g., Clq) followed by fluorescent or enzyme conjugated polyclonal anti-Clq. Since there are many epitopes on a single Clq that may be recognized by the polyclonal antibody, the resulting signal will be amplified several fold over that attainable by labeling Clq itself.

Alternatively, binding of the detection reagent (e.g., Clq) may be visualized by changes in surface plasmon resonance. As the molecular layer becomes thicker with the sequential binding of antigen, Clq, (with or without polyclonal anti-Clq to further amplify the signal), the light refractive properties of the layer change and can be measured with appropriate instrumentation.

### G. Uses of the Invention

The invention is a general method for detecting ligand binding. Since binding reactions, such as, antibody recognition of antigens, is widely used in the quantitation of proteins and other molecules, the detection of such complexes is important for many applications, including (but not limited to) proteomics and diagnostic measurements of proteins and other molecules in bodily tissues and fluids.

The technology can be used for detecting any molecule that is capable of acting as an antigen or hapten in binding to an antibody. This includes applications for the detection of pesticides, pathogens, and other contaminants in water or air for environmental testing, in animal or human sera or tissue samples for the diagnosis of disease or for assessing the biological state of an organism, for validation of therapeutic drugs where biological markers are available for assessing potential side-effects in new drugs, for high-throughput screening of compounds for therapeutic applications, and any other application where detection or quantitation of a molecule in a heterogeneous population of molecules is desired.

All patents and publications referred to herein are expressly incorporated by reference in their entirety. The following examples serve to illustrate certain embodiments and aspects of the present invention and are not to be construed as limiting the scope thereof.

### EXAMPLES

### Example 1

Hybridomas producing high affinity antibodies for a specific antigen are selected by standard techniques. The variable regions of the Ig heavy and light chains expressed by this hybridoma are cloned and introduced into IgM expression vectors by standard techniques. This vector allows the fusion of these variable regions into the appropriate light chain gene (kappa or lambda) and the IgM heavy chain gene. These DNA constructs are then introduced into a cell appropriate for their expression and secretion, such as a myeloma cell line. The fusion IgM molecules produced by these cells are purified by standard means and immobilized on a slide again by standard means.

A sample (tissues or body fluids) is then allowed to react on the surface of the slide and antigen in the sample binds to the antibody. The antibody:antigen complexes are then detected with a reagent with coupled Clq. This may be a fluorescent compound, or a macromolecule large enough to provide a change in optical properties such as refractive index or light scattering.

### Example 2

This example uses the same protocol as described in Example One, however, the Clq binding site in the IgM expression vector has been altered. Antibodies are then raised to this novel IgM site, accessible only when antigen is bound to the mutant IgM, These antibodies can then be coupled in the same way as Clq, such as to a flourescent compound or to a macromolecule large enough to provide a change in optical properties such as refractive index or light scattering.

### Example 3

The class of soluble bacterial receptors exemplified by the sensor components of bacterial two-component signal transduction systems (TCSTS). Certain of the sensor components of TCSTSs (e.g., E. coli NtrB) are soluble cytoplasmic proteins composed of two domains: a ligand binding domain and an effector (or “transmitter”) domain. The sequences of the ligand binding domains are quite different among the members of this class of protein, reflecting the specificity of each protein for binding a single specific ligand. The sequences of the effector domains, however, are highly conserved, and have protein kinase activity that is activated upon ligand binding to the ligand-binding domain. Thus the activation of the effector domain can be accomplished by ligand binding.

### Example 4

This example uses the fusion IgM molecules made in Example One. The IgM molecules are allowed to react with a sample containing the ligand for the IgM. When the IgM molecules bind their cognate ligands, they undergo a conformational change. The conformational change is detected by physical means such SPR.

### Example 5

A high affinity IgM AviTagged antibody capable of Universal Detection was constructed from an existing hybridoma cell line producing antibody recognizing the hepatitis B Surface antigen (HBsAg). In order to construct an IgM molecule from antibodies of a different class (e.g., IgG, IgA, IgE, IgD, IgY) or from different species such as llama or chicken, or from binding regions of Ig-like molecules (such as, but not limited to, T-cell receptors) it is first necessary to construct a vector containing the IgM constant regions to which the various variable domains can be appended. This parental vector was constructed in Invitrogen's FLP-INM vector by PCR of DNA from a murine spleen cDNA library and blunt-end cloning into a sequencing vector such as—TOPO® vector from Invitrogen (Cat: 45-0030) using the manufacturer's protocol.

PCR primers for cloning the IgM constant regions were designed around the sequence of the constant heavy chains to allow sub-cloning into the Flp-In™ vector from Invitrogen. They were designed with and without a STOP codon so that the AviTag could be added to the 3′ end. Upon verification that the IgM constant regions were cloned correctly, the DNA sequence encoding the AviTag biotin-accepting peptide was cloned in-frame using techniques familiar to those skilled in the art.

To append the IgG variable regions for HBsAg to the IgM backbone, it is necessary to make cDNA containing the IgG variable regions (heavy and light chains) from the hybridoma producing the IgG HBsAg antibody. The mRNA form the HbSAg hybridoma was extracted and pelleted via the following protocol:

A. RNA Extraction from the anti-HBsAg hybridoma. 15 million hybridoma cells were pelleted and washed twice with phosphate-buffered saline (PBS).

The cells were resuspended in 1 ml of STAT60™ RNA extraction reagent (BioGenesis Cat: CS-110), incubated for 5 minutes at room temperature and 20 μl of chloroform added. The suspension was shaken vigorously for 15 seconds and incubated at room temperature for 3 minutes prior to centrifugation at 12000 rpm for 5 minutes at 4° C.

This formed 2 phases: a lower, red, phenol chloroform phase; and an upper colourless phase, containing the RNA. The interphase contains both DNA and proteins.

The upper, aqueous layer was mixed with 0.5 ml of isopropanol and incubated for 10 minutes at room temperature prior to centrifugation at 12000 rpm for 10 minutes at 4° C.

The RNA pellet was washed in 75% ethanol and centrifuged at 7500 rpm for 5 minutes at 4° C. The pellet was dried and dissolved in 80 μl of RNAse free water.

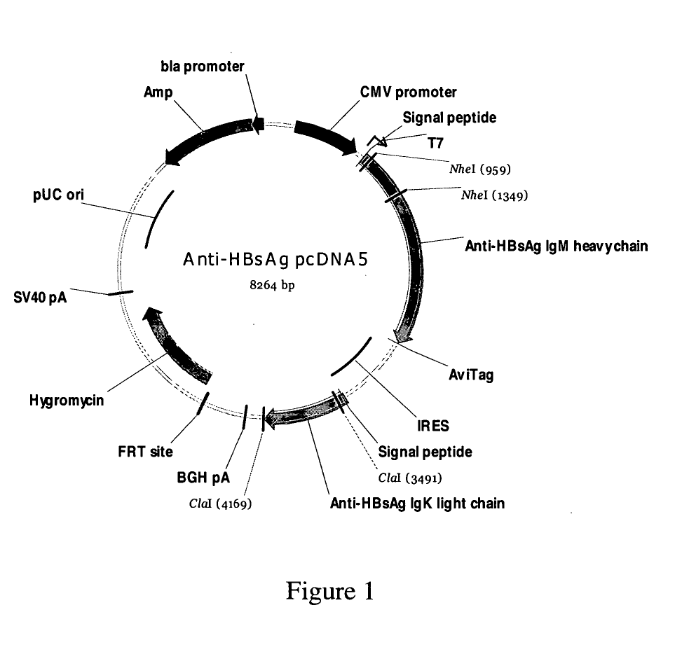

B. Cloning the Ig Specific DNA. cDNA was synthesized from the purified RNA by RT-PCR using the ImProm-II™ reverse transcription kit from Promega (Cat: A3800) according to the manufacturer's instructions. The Ig variable regions of the IgG heavy chains, and the entire IgG kappa light chain comprising both the constant and heavy regions, were cloned by PCR of the cDNA using Ig specific variable heavy and variable light chain primers from Amersham Pharmacia (Heavy primers Cat: 27-1586-01; Light primers Cat: 27-1583-01), following the manufacturer's protocol. PCR products were cloned into the pCR4-TOPO vector from Invitrogen (Cat: 45-0030) using the manufacturer's protocol and sequenced. Ig positive DNA sequences were PCR amplified from the TOPO vector using specific primers including restriction sites for sub-cloning into the Flp-In™ vector. The VH sequence was cloned by cleavage of the heavy-chain variable region PCR product with Nhe1 restriction endonuclease, and the light chain PCR product was cloned using Clal restriction endonuclease following standard protocols. The final expression vector is shown schematically in FIG. 1. The Anti-HBsAG expression vector was transfected into Flp-In™ CHO cells for the generation of stable mammalian protein expression cell lines. For the HbSAg, the Variable Light chain is cloned into the vectors using ClaI restriction endonuclease sites, while the Variable Light chain is cloned into the expression vector using NheI sites.

C. Generation of stable Flp-In™ cell lines. Flp-In™ CHO cells were grown routinely in Zeocin™ medium before being split into P90 tissue culture plates at a density of 1.5×106 cells/ml in 10 mls of Zeocin™ growth medium. Cells were incubated for 24 hours before transfection using FuGENE™ 6.

D. Transfection. 87.5 μl of serum free Hams F12 nutrient mix medium and 7.5 μl of FuGENE™, were sequentially aliquoted into 4 sterile Eppendorf microtubes, For each transfection 0.5 μl of the vector DNA coding for Avitagged anti- HepB IgG and IgM in pcDNA 5/FRT, empty vector control or serum free medium control, were added to the FuGENE™ serum free medium mixes. To all the transfection mixes 4.51 μl of pOG44:pcDNA5/FRT plasmid DNA was added, transfection mixes were tapped gently to mix and incubated at room temperature for 30 minutes.

5 mls of medium was aspirated from the Flp-In™ CHO cells, and the appropriate transfection mix was carefully added to the culture medium. Transfected cells were incubated overnight. 24 hours after transfection, medium was removed from the cells and replaced with10 mls of fresh Zeocin growth medium.

48 hours following transfection cells were split into Hygromycin B containing growth medium at <25% confluency. Cells were further cultured replenishing the Hygromycin medium every 2-3 days until foci can be identified. 20 hygromycin foci are isolated and the cells expanded. Cells were then checked for integration of the pcDNA/5FRT construct by testing for Zeocin sensitivity. Clones are then further examined for expression of the Avitagged antibodies.

E. Cell Culture/media. Flp-In™ Chinese Hamster Ovary (CHO) and mCAT CHO cells were maintained in, Hams F12 Nutrient Medium, supplemented with 10% foetal calf serum, L-glutamine (2 mM), Penicillin (1.0 IU ml⁻¹) and Streptomycin (1.0 mg ml⁻¹). Additionally medium for Flp-In™ CHO cells contained 100 μg/ml of Zeocin, while mCAT CHO cell medium contained 300 μg/ml of Hygromycin B.

Plat.-E cells were maintained in Dulbecco modified medium supplemented with 10% fetal calf serum, L-glutamine (2 mM), Penicillin (1.0 IU ml⁻¹) and Streptomycin (1.0 mg ml⁻¹).

Unless stated, cells were incubated at 37° C., 6% CO₂, and all cell culture preparatory procedures were carried out in a laminar flow biological safety cabinet under aseptic conditions.

### Example 7

Instead of using existing hybridomas as in example 6, the IgM antibody for a Physically Alterable Binding Reagent could be constructed from available antibody DNA sequences. The HBsAg antibody DNA sequence is posted in the NCBI database. The sequence describes a scFv (single chain Fv antibody) sequence deposited in the NCBI database (Accession No.: AF236816).

Variable heavy chain domain:

| | |
|---|---|
| Variable heavy chain domain: | |
| atggccgaggtgcagctggtggagtctgggggaggct | (SEQ ID NO:3 |
| tggtcaagcctggagggtccctgagactctcctgtgc | SEQ ID NO:5) |
| agactctggattcaccttcagtgactactacatgagc | |
| tggatccgccaggctccagggaaggggctggagtggg | |
| tttcatacattagtagtagtggtagtaccatatacta | |
| cgcagactctgtgaagggccgattcaccatctccagg | |
| gacaacgccaagaactcactgtatctgcaaatgaaca | |
| gcctgagagccgaagacacggccgtgtattactgtgc | |
| aagaaagctgaggaatgggaggtggcctctggtttat | |
| tggggccaaggtaccctggtcaccgtgtcgaga; | |
| translated protein sequence,. | |

Variable light chain domain:

| | |
|---|---|
| Variable light chain domain: | |
| tctgagctgactcaggaccctgctgtgtctgtggcct | (SEQ ID NO:6 |
| tgggacagacagtcaggatcacatgccaaggagacag | SEQ ID NO:8) |
| cctcagaagctattatgcaagctggtaccagcagaag | |
| ccaggacaggcccctgtacttgtcatctatggtaaaa | |
| acaaccggccctcagggatcccagaccgattctctgg | |
| ctccagctcaggaaacacagcttccttgaccatcact | |
| ggggctcaggcggaagatgaggctgactattactgta | |
| actcccgggacagcagtggtaaccatgtggtattcgg | |
| cggagggaccaagctgaccgtcctaggtgcggccgca | |
| gaacaaaaactcatctcagaagaggatctgaatgggg | |
| ccgcatag; | |
| translated protein sequence,. | |

Unlike IgM class antibodies, single chain antibodies are not suitable for use as a Physically Alterable Binding Reagent because they are not known to undergo the conformational change (analougous to IgM) upon ligand binding; however, an AviTagged IgM antibody suitable use as a Physically Alterable Binding Reagent could be constructed using the variable heavy chain domain sequence and a light chain domain sequence (variable and constant regions) that together contain the HBsAg antibody binding domains. As in Example 6, DNA encoding the heavy chain variable region of an immunoglobulin, or other protein binding domain, is cloned in-frame into a vector containing the IgM constant region and co-expressed with the cloned immunoglobulin light chain using methods known to those skilled in the art. DNA encoding the AviTag biotin-accepting peptide would be cloned to the C-terminus of the IgM heavy chain to allow the Physically Alterable Binding Reagent antibodies to be immobilized onto surfaces or molecules using the biotin/streptavidin interaction.

Oligonucleotides of these variable heavy and light chain DNA sequences could synthesized with ends suitable for ligation into an expression vector designed for the stable integration and expression of proteins. Because of the length of the DNA sequences, multiple complimentary oligos could be synthesized that overlap in sequence and annealed and extended using a thermophilic polymerase, using the technique “jump-PCR”.

### Example 8

As an extension of the use of IgM-like molecules as Physically Alterable Binding Reagent, certain species of animals (e.g., llama and sharks) are known to produce classes of immunoglobulin or Ig-like molecules, devoid of a light chain, in which the antigen-combining site is derived from only the heavy chain. An IgM-like chimeric Physically Alterable Binding Reagent molecule can be constructed by recombinant DNA techniques using the IgM mu chain constant region backbone described elsewhere in this application, joined to the complete variable region antigen combining site contained on the heavy chain of these single chain antibodies. Likewise, scFv (e.g., from phage display technologies) are immunoglobulin-derived molecules, in which the light and heavy chain-derived variable regions are contiguous on a single polypeptide chain. Together, these two contiguous variable regions constitute the functional antigen-combining site. An IgM-like chimeric Physically Alterable Binding Reagent molecule can be constructed by recombinant DNA techniques using the IgM mu chain constant region backbone described elsewhere in this application, joined to the complete, contiguous variable region antigen combining site contained on an scFv.

### Example 9

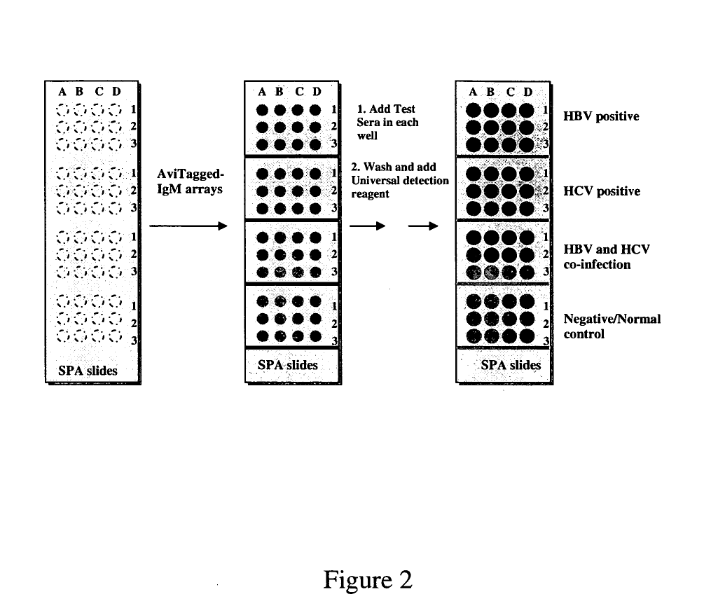

Multiple Physically Alterable Binding Reagents, each with a unique antigen specificity can be immobilized to a solid support in an addressable array format. This allows simultaneous detection of each antigen in a single test. An array can be constructed to simultaneously and separately detect the presence of multiple viral pathogens in the serum of humans and animals. Referring to FIG. 2, four AviTagged IgM Physically Alterable Binding Reagent reagents specific for HIV (column A), HBV (column B), HCV (column C) and SARS (column D), respectively, are arrayed in duplicate (rows 1 and 2). Total IgM isolated from non-immune normal human blood serves as a control spot for each column (row 3). Each group of these 12 spots is iterated multiple times on the slide (four in this example) to allow simultaneous testing of multiple patient sera. Each group of arrays on the slide (rows 1-3) is separated by leak-proof plastic dividers to physically separate the patients' samples. The test sera and normal sera will be loaded and respective virus that may be present in the patient sera will be captured by the specific IgM Physically Alterable Binding Reagents. The conformational change in Physically Alterable Binding Reagent occurs upon binding of the viral particles, and detected using the Universal Detection Reagent.

### Example 10

Commercially available IgM antibodies for either E. coli beta galactosidase or for mammalian c-erbB3 were coupled to the wells of a 96 well microtiter tray through the Pierce maleimide coupling. Non-specific sites on the plates were then blocked by treatment with bovine serum albumin in saline. Three different dilutions of commercially available beat galactosidase were added to wells with the anti-beta galactosidase antibodies and with the anti-c-erbB3 antibodies. The unbound beta galactosidase was then washed from the wells, and fluorescently labeled Clq was added. After washing the unbound Clq from the plates, the amounts of bound Clq were determined by measuring the fluorescent label. There were two salient results: First, with low ionic strength binding buffer, the wells containing the c-erbB3 antibody (non-specific) showed no signal over background. The wells containing anti-beta galactosidase antibodies gave significant signal over background that depended on antigen (i.e. beta-galactosidase) varied with the Clq concentration. These results show that Clq binding can be used with certain linking arrangements of antibodies to detect the amount of antigen bound, and hence the concentration of an antigen in a sample.

### Example 11

The specific Physically Alterable Binding Reagent and antigen complex may also be formed in solution. In this example, IgM Physically Alterable Binding Reagent is added to a test sample wherein specific antigen is bound to the Physically Alterable Binding Reagent. Clq is added to the mixture to form Physically Alterable Binding Reagent-antigen-Clq ternary complex. This complex is captured by an IgM specific reagent [e.g., anti-IgM F(ab)₂ ] immobilized on a solid support. The unbound Clq and other components are removed by washing, and the Clq contained in the complex is detected using Cy5-labeled anti-Clq antibody.

1. A method for detection of binding, comprising:
a) providing a Physically Alterable Binding Reagent,
b) providing a ligand, wherein the Physically Alterable Binding Reagent specifically binds to the ligand;
c) detecting a conformational change in the Physically Alterable Binding Reagent, whereby binding of the Physically Alterable Binding Reagent to the ligand is detected.

2. A method for detection of binding, comprising the method of claim 1, wherein said detecting a conformational change in the Physically Alterable Binding Reagent comprises:
a) providing a Universal Detection Reagent; and
b) detecting the binding of the Universal Detection Reagent to the Physically Alterable Binding Reagent, whereby binding of the Physically Alterable Binding Reagent to the ligand is detected.

3. The method of claim 1, wherein the detection is quantitative.

4. The method of claim 1, wherein the Physically Alterable Binding Reagent comprises
a) a ligand binding site;
b) a domain that becomes physically altered upon binding of ligand to the ligand binding site: and,
c) optionally, a site useful for coupling the binding reagent to a solid support.

5. The method of claim 1, wherein the Physically Alterable Binding Reagent comprises an antibody or a receptor binding-domain.

6. The method of claim 5, wherein the antibody comprises an antibody selected from the group consisting of monomeric IgM, oligomeric IgM, an Fab fragment, an F(ab)₂ fragment, a genetically engineered antibody and a chimeric antibody.

7. The method of claim 1, wherein the Physically Alterable Binding Reagent comprises a tag for coupling the Physically Alterable Binding Reagent to a solid support.

8. The method of claim 7, wherein the tag is selected from the group consisting of biotin accepting peptide sequence, hexa-His peptide, Strep-Tag, Strep-TagII, FLAG, epitope tag, maltose binding protein (MBP), glutathione-S-transferase (GST), green fluorescent protein (GFP), red fluorescent protein (RFP), blue fluorescent protein (BFP), chitin binding protein, calmodulin binding protein (CBP), cellulose binding domain, S-tag, FIAsH, RsaA, and sortase recognition sequence.

9. The method of claim 8, wherein the biotin accepting peptide sequence is LeuXaa₁Xaa₂IleXaa₃ Xaa₄Xaa₅Xaa₆LysXaa₇Xaa₈Xaa₉Xaa₁₀, where Xaa₁ is any amino acid; Xaa₂ is any amino acid other than Leu, Val, Ile, Trp, Phe, or Tyr; Xaa₃ is Phe or Leu; Xaa₄ is Glu or Asp; Xaa₅ is Ala, Gly, Ser, or Thr; Xaa₆ is Gln or Met; Xaa₇ is Ile, Met, or Val; Xaa₈ is Glu, Leu, Val, Tyr, or Ile; Xaa₉ is Trp, Tyr, Val, Phe, Leu, or Ile; and Xaa₁₀ is any amino add other than Asp or Glu, wherein said biotinylation-peptide is capable of being biotinylated by a biotin ligase at the lysine residue adjacent to Xaa₆.

10. The method of claim 9, wherein the biotin accepting peptide sequence is SEQ ID NO:2.

11. The method of claim 8, wherein said biotinylation sequence has been biotinylated by a biotin ligase.

12. The method of claim 3, wherein the Universal Detection Reagent selected from the group consisting of Clq, Clq binding site-specific antibody, and an anti-J chain-specific antibody.

13. The method of claim 3, wherein the Universal Detection Reagent comprises a reporter molecule.

14. The method of claim 13, wherein the reporter molecule is selected from the group consisting of an enzyme that reacts with a substrate to give distinctive product, a fluorescent dye, and a gold particle.

15. The method of claim 1, wherein detecting a conformational change in the Physically Alterable Binding Reagent to the ligand is selected from the group consisting of fluorescence emission, Raman shift spectroscopy, Fluorescence Resonance Energy Transfer (FRET), Surface Plasmon Resonance, and Atomic Force Microscopy.

16. A method for detection of binding, comprising:
a) providing an antibody;
b) providing a ligand, wherein the antibody specifically binds to the ligand;
c) providing a Universal Detection Reagent; and
d) detecting the binding of the Universal Detection Reagent to the Physically Alterable Binding Reagent, whereby binding of the Physically Alterable Binding Reagent to the ligand is detected.

17. The method of claim 16, wherein the antibody comprises an IgM portion, and wherein the Universal Detection Reagent comprises Clq comprising a reporter molecule.

18. The method of claim 17, wherein the antibody comprises an IgM portion, and wherein the Universal Detection Reagent is selected from the group consisting of Clq, Clq binding site-specific antibody, and an anti-J chain-specific antibody.

19. A microarray comprising a plurality of Physically Alterable Binding Reagents at specific locations on the surface of said solid support in an addressable format.

20. A method for detecting binding, comprising:
a) preparing a microarray according to claim 19;
b) providing a sample suspected of ligand containing a ligand, wherein the Physically Alterable Binding Reagent specifically binds to the ligand;
c) providing a Universal Detection Reagent; and
d) detecting the binding of the Universal Detection Reagent to the Physically Alterable Binding Reagent, whereby binding of the Physically Alterable Binding Reagent to the ligand is detected.
